(19) United States
(12) Patent Application Publication
(10) Pub. No.: US 2010/0272708 A1
(43) Pub. Date: Oct. 28, 2010

(54) METHOD FOR THE TREATMENT OF TYPE AND TYPE IV HYPERSENSITIVITY

(76) Inventors: Christina Juneau, Tewkesbury (CA); Rejean Drouin, Quebec (CA); Olivier Moroni, Quebec (CA)

Correspondence Address: OGILVY RENAULT LLP, 1, Place Ville Marie, SUITE 2500, MONTREAL, QC, H3B 1R1, CA

(21) Appl. No.: 12/599,905

(22) PCT Filed: Jun. 10, 2008
(86) PCT No.: PCT/CA2008/001158

§ 371 Date: Nov. 12, 2009

Related U.S. Application Data
(60) Provisional application No. 60/943,073, filed on Jun. 11, 2007.

(51) Int. Cl. A61K 38/18; A61K 39/395; A61P 17/00

(52) U.S. Cl. 424/130.1; 514/8.9

(57) ABSTRACT

The present application relates to methods for the treatment of eczema and uses of a dairy-derived composition. The treatment involves oral administration of 800 mg/day of a composition comprising TGF-β1, TGF-β2, IGF-1 and 60% (w/w) beta-lactoglobulin derived from whey.

### FIELD OF INVENTION

The present invention relates in general to the field of the treatment of allergies and atopic disorders (Type I and IV hypersensitivity), and more particularly to an oral composition that includes a pharmaceutically effective amount of bio-active peptides and proteins from dairy sources for the treatment of Type I and IV hypersensitivity-related disorders.

### BACKGROUND

### Immune Response Associated with Type I and IV Hypersensitivity

The immune system is a very complex and well organized system that protects the body against infectious organisms and other invaders. The goal of the immune system is to attack, neutralize and destroy foreign organisms and substances that invade our systems that may cause illness. The immune system has evolved to mount robust responses to pathogens while maintaining tolerance to self or innocuous antigens as those in commensal bacteria. However, in some cases the immune response can be inadequate and lead to uncontrolled immune responses like inappropriate inflammation or immune related diseases like psoriasis, allergies, inflammatory bowel diseases, arthritis, HIV, lupus, atopic dermatitis (eczema) to name only those.

The immune system is made up of a network of cells, tissues, and organs that work together to protect the body against potential invaders. The cells that are part of this defense system are white blood cells, or leukocytes. Leukocytes come in two basic types; (1) the phagocytes are cells that chew up invading organisms and (2) the lymphocytes are cells that allow the body to remember and recognize previous invaders.

There are two kinds of lymphocytes: the B lymphocytes and the T lymphocytes. T lymphocytes play a central role in cell-mediated immunity. The cell-mediated immune system consists of T-cells which originate in the bone marrow, but go to the thymus to finish their development. T-cells are highly-specialized cells in the blood and lymph to fight bacteria, viruses, fungi, protozoans, cancer, etc. within host cells and react against foreign matter such as organ transplants.

Several different subsets of T cells have been described, each with a distinct function. (1) Cytotoxic T cells (Tc cells) destroy virally infected cells and tumor cells, and are also implicated in transplant rejection. These cells are also known as CD8+ T cells, since they express the CD8 glycoprotein at their surface; (2) Helper T cells, (Th cells) are the “middlemen” of the adaptive immune system. Once activated, they divide rapidly and secrete small proteins called cytokines that regulate or “help” the immune response. These cells (also called CD4+ T cells) are a target of HIV infection; the virus infects the cell by using the CD4 protein to gain entry. (3) Memory T cells are a subset of antigen-specific T cells that persist long-term after an infection has resolved. They quickly expand to large numbers of effector T cells upon re-exposure to their cognate antigen. Memory cells may be CD4+ or CD8+; (4) Regulatory T cells (Treg cells), formerly known as suppressor T cells, are crucial for the maintenance of immunological tolerance. Their major role is to shut down T cell mediated immunity towards the end of an immune reaction and to suppress auto-reactive T cells that escaped the process of negative selection in the thymus. (5) Natural Killer T cells (NKT cells) are a special kind of lymphocyte that bridges the adaptive immune system with the innate immune system. Once activated, these cells can perform functions ascribed to both Th and Tc cells (i.e. cytokines production and release of cytolytic/cell killing molecules).

The helper T cell responses to antigen can be generally divided into distinct types based on cytokines expression; designated Th1 and Th2. Hallmark cytokines expressed by Th1 cells are IFN-γ and IL-2 and Th2 cells express IL-4. IL-2 is important for T-cell growth and activation while IFN-γ induces expression of MHC class I and II molecules, activates monocytes and macrophages, and favors the development of Th1 cells. The Th2 cytokines have a crucial role in the initiation of the allergic response. IL-4 is important for IgE production, development of Th2 cells, and induction of adhesion molecules on endothelial cells that recruit eosinophils (Romagnani, 2000).

B-cells, the other class of lymphocytes, originate in the bone marrow and stay there to develop. B-cells can produce immunoglobulins (antibodies), but need exposure to foreign antigens to do so. Immunoglobulins (Ig) are proteins in blood plasma and lymph to fight bacteria and viruses in body fluids. All daughter cells of a B-cell will be able to produce the same Ig as the mother cell. Ig binds to certain parts of an antigen to mark it for destruction (by the T-cells). In mice and humans, the different types of antibody are known as IgM, IgG, IgA, IgD, and IgE. In addition, there are 4 IgG and 2 IgA subtypes present in humans. Each immunoglobulin class differs in its biological properties and locations, and has evolved to deal with different antigens.

In biology, IgE is an antibody subclass (known as “isotypes”), found only in mammals. IgE is capable of triggering the most powerful immune reactions. Most of our knowledge of IgE has come from research into the mechanism of a form of allergy known as type 1 hypersensitivity. IgE production by B cells requires a physical interaction with T cells, involving a number of surface adhesion molecules, as well as the soluble factors IL-4 and IL-13 produced by T cells, basophils and mast cells (Maggie, 1998).

Type I hypersensitivity disorders (allergic rhinitis, asthma, systemic anaphylaxis) are associated with antibodies production (IgE and IgG) and are distinguished by the different types of antigens recognized at the different classes of antibody involved. Type I hypersensitivity reactions underlie all atopic and many allergic disorders. The terms atopy and allergy are often used interchangeably but are different. Atopy is an exaggerated IgE-mediated immune response; all atopic disorders are type I hypersensitivity disorders. Allergy is any exaggerated immune response to a foreign antigen regardless of mechanism. Thus, all atopic disorders are considered allergic, but many allergic disorders (e.g., hypersensitivity pneumonitis) are not atopic. Allergic disorders are the most common disorders among people (Janeway et al. 2001).

Type IV hypersensitivity reactions are T cell-mediated (Th-1 and Th-2) and can lead to T cell-dependent diseases like contact dermatitis, tuberculin reaction, chronic asthma, chronic allergic rhinitis. Unlike the immediate hypersensitivity reactions which are mediated by antibodies, the delayed-type or type IV hypersensitivity reactions are mediated by antigen-specific effector T cells. Delayed-type hypersensitivity, contact hypersensitivity and gluten-sensitive enteropathy (celiac disease) are the three groups of symptoms related to type IV hypersensitivity. These reactions are mediated by T cells (Janeway et al. 2001).

### Modulation of Immune Function by Whey Protein-Derived Products

The nutritional benefits of bovine milk and milk-related products have been acknowledged and used, arguably, for centuries. It is now known that bovine milk contains several components that can modulate immune functions and recent research has demonstrated that some individual components of milk, purified to a high degree, can directly affect cells of the immune system (Cross & Gill 1999, 2000). Commercial attention is now being paid to the characterization of dairy components that modulate immune functions, in order to exploit their potential benefits in human health improvements (Marshall 2004). It is known that beta-and kappa-casein (in particular the carbohydrate-rich glycomacropeptide component of the latter) has a variety of suppressive and stimulatory effects on mononuclear cell functions (Otani and Monnai 1993, Wong et al. 1996). In addition, some of the proteins isolated and purified from whey have been shown to be potent modulators of cellular immune functions; these include lactoperoxidase, lactoferrin, and milk growth factors (e.g. TGF-β) (Otani and Odashiman 1997, Wong et al. 1997). In general, the immunomodulatory properties of these individual components become more evident (and the dose response more potent) as they are progressively purified from whole casein or whey.

The essential role of milk-derived immunosuppressive factors during early development has been demonstrated in the murine system by Kulkarni and Karlsson (1993), who have shown that neonatal transforming growth factor (TGF)-β-deficient mice remain viable only as long as they receive maternal milk containing TGF. TGF-β is a potent regulator cytokine with diverse effects on hemopoietic cells. The pivotal function of TGF-β in the immune system is to maintain tolerance via the regulation of lymphocyte proliferation, differentiation, and survival. Collectively, TGF-βs inhibit the development of immunopathology to self or non-harmful antigens without compromising immune response to pathogens (Li et al. 2006).

TGF-β exerts the greatest impact on T lymphocytes. TGF-β inhibits T cell proliferation via the inhibition of IL-2, likely via suppression of IL-2 transcription (Kehrl et al. 1986). TGF-β is also a potent regulator of effector T cell differentiation, and it generally inhibits the acquisition of Th cell functions (Gorelick & Flavell 2002). TGF-β also regulates cytotoxic T lymphocytes differentiation (CD8+) (Li et al. 2006). CD8+ T cell exert their effector functions through production of inflammatory cytokines such as IFN-γ. In addition to proliferation, TGF-β potently inhibits CD8+ T cell differentiation (Li et al. 2006). TGF-β is an important regulator of B lymphocyte activity. TGF-β inhibits B cell proliferation, induces apoptosis of immature resting B cells, and blocks B cell activation and class switching to most isotypes except for IgA (Li et al. 2006). Thus similar to T cell, TGF-β has both inhibitory and stimulatory effects on B cells.

U.S. Pat. No. 7,141,262 and WO 07/038,870 disclose dairy-derived compositions that are used for the treatment of inflammatory disorders and/or psoriasis.

It is desirable to provide an effective, well tolerated composition that can be used in the treatment of type I and type IV hypersensivity, and more particularly for the treatment of eczema.

### SUMMARY OF THE INVENTION

It has now been found that the present invention provides a composition for the prophylaxis or treatment of allergies or atopic disorders or related disorders, the composition comprising between 0.1 to 5 μg of TGF-β1 per gram of composition, between 5 to 50 μg of TGF-β2 per gram of composition, and at least 60% of β-lactoglobulin. Alternatively, TGF-β1 and TGF-β2 can be dairy derived, and can be found at concentration between 0.2 to 1.2 μg/g and between 8 to 18 μg respectively per gram of composition.

Another aim of the present invention is to provide a method for treating allergies or atopic disorders or related disorders in a patient in need thereof, the method comprising administering to the patient an effective amount of the composition as defined above.

Another aim of the present invention is to provide a method for preventing recurrences of allergies or atopic disorders or related disorders in a patient in need thereof, the method comprising administering to the patient an effective amount of the composition as defined above.

Accordingly, one aspect of the present invention contemplates a method for ameliorating the effects of a proliferative and/or inflammatory skin disorder associated with allergies or atopic disorders in a mammal, the method comprising treatment of a subject affected by the allergies or atopic disorders and related diseases with an effective amount of the composition as defined above.

Accordingly, a further aspect of the present invention contemplates a method for treating atopic dermatitis (eczema) and related diseases in a mammal, the method comprising orally or topically administering to the mammal an effective amount of the composition as defined above.

According to a further aspect of the invention, there is provided the use of a composition comprising between 0.1 to 5 μg of TGF-β1 per gram of composition; between 5 to 50 μg of TGF-β2 per gram of composition, and at least 60% of β-lactoglobulin for the manufacture of a preparation for the prophylaxis or treatment of allergies or atopic disorders or related disorders

Particularly, TGF-β1 and TGF-β2 can be dairy derived, and can be found at concentration between 0.2 to 1.2 μg/g and between 8 to 18 μg respectively per gram of composition.

Particularly, the invention relates to the use of the above-described composition for the manufacture of a preparation for the treatment of allergies or atopic disorders or related disorders.

Still, particularly, the invention relates to the use of the above-described composition for the manufacture of a preparation for the prevention of recurrences of allergies or atopic disorders or related disorders.

More particularly, the invention relates to the use of the above-described composition for the manufacture of a preparation for ameliorating the effects of a proliferative and/or inflammatory skin disorder associated with allergies or atopic disorders.

Even more particularly, the invention relates to the use of the above-described composition for the manufacture of an oral or topical preparation for the treatment of atopic dermatitis (eczema) and related diseases.

Someone skilled in the art will recognize that types of allergies, atopic disorders or related disorders that can be prevented or treated with the method and uses of the present invention may include: allergic rhinitis, asthma, systemic anaphylaxis, and T cell-dependent diseases like: contact dermatitis (eczema), tuberculin reaction, chronic asthma, and chronic allergic rhinitis. (HIVES, urticaria)

### DETAILED DESCRIPTION OF THE INVENTION

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

### BRIEF DESCRIPTION OF THE DRAWINGS

FIG. 1. A) Dose-dependent inhibition of Concanavalin A (ConA)-induced murine splenocytes proliferation by the composition of the present invention as described in example I. (SI=Stimulation Index) B) Dose-dependent inhibition of ConA-induced IL-10 production by the composition of the present invention as described in example I. C) Dose-dependent inhibition of ConA-induced murine IL-4 production by the composition of the present invention. * represent statistically significant data at p>0.05;

FIG. 2. A) Decreased proportion of CD8⁺ cells in presence of the composition (100 & 1000 μg/ml) as described in example I. B) Decreased ratio of CD8⁺/CD25⁺ cells in presence of the composition (100 & 1000 μg/ml). * represent statistically significant data at p>0.05;

FIG. 3. Decreased of ConA-induced murine splenocytes proliferation following 8 weeks of gavage with the composition (8, 80 & 400 mg/kg) in healthy mice;

FIG. 4. Decreased of ConA-induced IL-4 production following 8 weeks of gavage with the composition (400 mg/kg) in healthy mice;

FIG. 5 shows the EASI scores of 9 patients treated with 800 mg/day of the composition at day 1, 30, 60 and 90 after commencement of treatment; and

FIG. 6 shows the DLQI scores of 9 patients treated with 800 mg/day of the composition at day 1, 30, 60 and 90 after commencement of treatment.

For the purpose of the present invention the following terms are defined below.

### DEFINITIONS

The term “about” as used hereinbelow refers to a margin of + or −5% of the number indicated. For sake of precision, the term about when used in conjunction with, for example, 90%, means 90%+/−4.5% i.e. from 86.5% to 94.5%.

The expression “effective amount” as used herein is intended to mean an amount sufficient to induce a beneficial or desired clinical result. An effective amount can be administered in one or more doses. For purposes of this invention, an effective amount of growth factors and/or dairy derived proteins, or other composition is an amount that induces a treatment or prophylactic response against at least one atopic dermatitis (eczema) responsible factor.

The terms “polypeptide”, “peptide” and “protein” are used interchangeably to refer to polymers of amino acids of any length, and may be interrupted by non-amino acids.

The terms “individual” or “subject” treated according to this invention is a vertebrate, preferably a mammal, more preferably a human. Mammals include, but are not limited to, farm animals, sport animals, rodents, primates, and pets.

The term “minerals” or “ashes” as used herein is intended to mean salt constituents and minerals at concentrations normally found in dairy products. For example, there can be different concentration of calcium, phosphorus, magnesium, potassium, sodium, chloride, sulfur, and citric acid. This also means that trace elements known in the art as found in dairy products can be assimilated or inferred therein.

Other terms used in this disclosure are explained where they arise.

### DESCRIPTION OF EMBODIMENTS

All patents, patent applications, articles and publications mentioned herein, both supra and infra, are hereby incorporated herein by reference.

Milk proteins are naturally presents as follows:

| | grams/liter | % of total protein |
|---|---|---|
| Total Protein | 33 | 100 |
| Total Caseins | 26 | 79.5 |
| alpha s1 | 10 | 30.6 |
| alpha s2 | 2.6 | 8.0 |
| beta | 9.3 | 28.4 |
| kappa | 3.3 | 10.1 |
| Total Whey Proteins | 6.3 | 19.3 |
| alpha lactalbumin | 1.2 | 3.7 |
| beta lactoglobulin | 3.2 | 9.8 |
| BSA | 0.4 | 1.2 |
| Immunoglobulins | 0.7 | 2.1 |
| Proteose peptone | 0.8 | 2.4 |

### Whey Proteins

The proteins appearing in the supernatant of milk after precipitation at pH 4.6 are collectively called whey proteins. These globular proteins are more water soluble than caseins and are subject to heat denaturation. Native whey proteins have good gelling and whipping properties. Denaturation increases their water holding capacity. The principle fractions are β-lactoglobulin, α-lactalbumin, bovine serum albumin (BSA), and immunoglobulins (Ig).

β-Lactoglobulins: (MW—18,000; 162 residues). This group, including eight genetic variants, comprises approximately half the total whey proteins. (β-Lactoglobulin has two internal disulfide bonds and one free thiol group. The conformation includes considerable secondary structures and exists naturally as a noncovalent linked dimmer. At the isoelectric point (pH 3.5 to 5.2), the dimmer are further associated to octamers but at pH below 3.4, they are dissociated to monomers.

α-Lactalbumins: (MW—14,000; 123 residues). These proteins contain eight cysteine groups, all involved in internal disulfide bonds, and four tryptophan residues. Alpha-Lactalbumin has a highly ordered secondary structure, and a compact, spherical tertiary structure. Thermal denaturation and pH<4.0 result in the release of bound calcium.

Enzymes: Enzymes are a group of proteins that have the ability to catalyze chemical reactions and the speed of such reactions. The action of enzymes is very specific. Milk contains both endogenous and exogenous enzymes. Exogenous enzymes mainly consist of heat-stable enzymes produced by psychotropic bacteria: lipases, and proteinases. There are many endogenous enzymes that have been isolated from milk. The most significant group is the hydrolases: lipoprotein lipase; plasmin; and alkaline phosphatase.

Lipoprotein lipase (LPL): A lipase enzyme splits fats into glycerol and free fatty acids. This enzyme is found mainly in the plasma in association with casein micelles. The milk fat is protected from its action by the fat globular matrix (FGM). If the FGM has been damaged, or if certain cofactors (blood serum lipoproteins) are present, the LPL is able to attack the lipoproteins of the FGM. Lipolysis may be caused in this way.

Plasmin: Plasmin is a proteolytic enzyme; it splits proteins. Plasmin attacks both (β-casein and alpha(s2)-casein. It is very heat stable and responsible for the development of bitterness in pasteurized milk and UHT processed milk. It may also play a role in the ripening and flavor development of certain cheeses, such as Swiss cheese.

Alkaline phosphatase: Phosphatase enzymes are able to split specific phosphoric acid esters into phosphoric acid and the related alcohols. Unlike most milk enzymes, it has a pH and temperature optima differing from physiological values; pH of 9.8. The enzyme is destroyed by minimum pasteurization temperature; therefore, a phosphatase test can be done to ensure proper pasteurization.

While a detectable immunological or tissue response is likely to be beneficial, efficacy can also be deduced by an improvement in symptoms or control of the disease condition beyond what would be expected without treatment.

The composition according to the invention is distinguished by strong anti-inflammatory and immuno-modulatory actions.

In accordance with the present invention, there is provided composition and methods for treating Type I or IV hypersensitivity disorders in an individual, comprising administering the composition effective in stimulating a specific immunological response against a physiological imbalance aberrantly expressed in Type I or IV hypersensitivity-related disorders. The composition comprises a cocktail of products that share growth and regeneration characteristics of a growth factor that is aberrantly expressed in Type I or IV hypersensitivity-related disorders. Particular growth factors included in, but are not limited to, the composition of the present invention, comprises transforming growth factors, such as TGF-β1 and TGF-β2, and at least 60% (w/w) of (β-lactoglobulin.

Preferably, the composition of the present invention will comprise at least 80% of dairy derived proteins. In one particular embodiment of the present invention, at least 70%, and preferably 80%, of the proteins are hydrosoluble. The proteins may be comprised, for example but not limited to, of between 0.1 to 30% (w/w) of lactoferrin.

In another embodiment of the present invention, the composition comprises at least 70% (w/w) of dairy derived proteins. The total concentration of proteins in the composition would normally be of at least 80% wherein at least 60% is β-lactoglobulin, preferably between 60% and 75%, most preferably between 65% and 70% of total proteins. Dairy proteins or dairy products as used herein may include milk, colostrums, or whey proteins or derived products or fractions thereof.

For prophylaxis, the active compounds are administered to a patient diagnosed with Type I or IV hypersensitivity, and prone to frequent relapse, in order to decrease manifestations of the disease in frequency and strength. Treatment in the manifest stage leads to its curtailment and to the alleviation of the symptoms.

In all types of Type I or IV hypersensitivity disorders, the active compounds of the present composition can be used prophylactically to avoid relapses or recurrences in affected patients and for the treatment of the disorders in acute phases.

The active compounds according to the invention can in particular also be used on skin superficially appearing to be healthy, e.g. in the case of allergies and atopy, i.e. also in addition to the diseased skin areas and, in particular, here too in the case of related Type I and IV hypersensitivity related disorders.

The present invention also addresses the underlying T-cell disorder that results in an inflammatory condition due to Type 1 or IV hypersensitivity disorders. The present inventors have recognized that most, if not all, of the current therapies for Type 1 or IV hypersensitivity disorders or similar T-cell mediated immune conditions are designed to modulate T-cells activity and thereby improve symptoms of inflammation or uncontrolled immune response. Without wishing to be bound by theory, it is possible that a major problem with the current treatments is that the therapy itself is so toxic that it may promote recurrence during healing. The toxicity of current treatments unleashes some or all of the cytokines that are associated with the promulgation of these chronic and often rebounding diseases.

The synergistic effects of the two TGF-β factors, β-lactoglobulin and other dairy-derived proteins result in a well tolerated, non-toxic, highly effective treatment for Type I and IV hypersensitivity disorders that is without the side effects observed with virtually all other therapies.

Likewise, the invention also relates to the use of the composition as oral or topical preparations. The invention also relates to the use of the active compound according to the invention for the production of pharmaceutical preparation, in particular oral, and cosmeceutical preparation, in particular topical, for the prophylaxis and treatment of Type 1 or IV hypersensitivity disorders.

The active compounds according to the invention can also be incorporated without problems into customary oral, pharmaceutical, dermatological, and cosmetic bases. The concentration of compounds in the composition of the present invention is adjusted depending of the needs. TGF-β1 can be found at concentrations of between 0.1 to 5 μg per gram of the composition and TGF-β2 can be found at concentration of between 5 to 50 μg per gram of the composition. Preferably, TGF-β1 is found at concentration of 0.2 to 1.2 μg/g, and TGF-β2 at between 10 to 18 μg/g of the composition. The preparations can be used daily in a customary manner. Advantageously, the composition can also comprise Insulin-like Growth Factor (IGF)-1.

In general, the composition of the present invention typically comprises:

| ingredient | Typical values | Window |
|---|---|---|
| TGF-β2 | 11.5 μg/g | 8 μg/g to 20 μg/g |
| TGF-β1 | 0.3 μg/g | 0.01 to 5 μg/g |
| IGF-1 | 0.1 μg/g | 0.01 to 2 μg/g |
| β-lactoglobulin (β-Lg) | 70% | 30% to 85% |
| α-lactalbumin (α-Lac) | 6% | 0% to 25% |
| Lactoferrin (Lf) | 4.5% | 0% to 15% |
| Immunoglobulin (IgG) | 5.5% | 2% to 15% |
| Hydrosolubility | 60% | Over 30% |
| Fat | 1% | 0% et 10% |

### Oral Administration

The active compounds according to the invention can be mixed with customary pharmaceutically acceptable excipients, diluents or vehicles and, if appropriate, with other auxiliaries and administered, for example, orally. They can preferably be administered orally in the form of granules, capsules, pills, tablets, film-coated tablets, sugar-coated tablets, syrups, emulsions, suspensions, dispersions, aerosols and solutions and also liquids. Preparations to be administered orally can contain one or more additives such as sweeteners, aromatizing agents, colorants and preservatives. Tablets can contain the active compound mixed with customary pharmaceutically acceptable auxiliaries, for example inert diluents such as calcium carbonate, sodium carbonate, lactose and talc, granulating agents and agents which promote the disintegration of the tablets on oral administration, such as starch or alginic acid, binding agents such as starch or gelatin, lubricants such as magnesium stearate, stearic acid and talc.

Suitable excipients are, for example, lactose, gelatin, maize starch, stearic acid, ethanol, propylene glycol, ethers of tetrahydrofurfuryl alcohol, microcrystalline cellulose, povidone, croscarmellose sodium, colloidal silicon dioxide, magnesium stearate, hydroxypropyl cellulose, and water.

The formulations are prepared, for example, by extending the active compounds with solvents and/or excipients, if appropriate using emulsifiers and/or dispersants, it being possible, for example, in the case of the use of water as a diluent optionally to use organic solvents as auxiliary solvents.

Administration is carried out in a customary manner, preferably orally, in particular perlingually or sublingually. In the case of oral administration, apart from the excipients mentioned, tablets, of course, can also contain additives, such as sodium citrate, calcium carbonate and dicalcium phosphate together with various additives, such as starch, preferably potato starch, gelatin and the like. Furthermore, lubricants such as magnesium stearate, sodium lauryl sulphate and talc can additionally be used for tableting. In the case of aqueous suspensions and/or elixirs, which are intended for oral administration, the active compounds can be mixed, apart from with the above-mentioned auxiliaries, with various flavors enhancers or colorants.

Capsules can contain the active compound as a single constituent or mixed with a solid diluent such as calcium carbonate, calcium phosphate or kaolin.

### Topical Administration

Ointments and topical formulations are particularly of interest also when considering the use of the composition of the present invention for treating contact dermatitis also called eczema.

The preferred compositions according to the invention can be formulated as liquid, pasty or solid preparations, for example as aqueous or alcoholic solutions, aqueous suspensions, emulsions, for example water/oil or oil/water emulsions, ointments, gels, lotions, creams, oils, powders or sticks. Depending on the desired formulation, the active compounds can be incorporated into pharmaceutical and cosmetic bases for topical application, which as further components contain, for example, oil components, fat and waxes, emulsifiers, anionic, cationic, ampholytic, zwitterionic and/or non-ionic surfactants, lower mono- and polyhydric alcohols, water, preservatives, buffer substances, thickeners, fragrances, colorants and opacifiers. Preferably, the emulsions (e.g. W/O emulsions, or ointments) are used.

Furthermore, it is preferred according to the invention to add antioxidants to the active compounds and to the pharmaceutical and topical preparations. The use of natural or naturally identical compounds such as, for example, tocopherols is particularly preferred here. The antioxidants mentioned are contained in the compositions according to the invention, for example, in amounts from 0.01-5% by weight, in particular from 0.5-2% by weight, based on the total composition.

### Dosage

The dosage administered will vary depending upon known factors, including but not limited to the activity and pharmaco-dynamic characteristics of the composition employed and its mode, time and route of administration; the age, diet, gender, body weight and general health status of the recipient; the nature and extent of the symptoms; the severity and course of the hypersensitivity reaction; the kind of concurrent treatment; the frequency of treatment; the effect desired. In general, the composition/preparation is most desirably administered at a dosage level that will generally afford immuno-modulatory results without causing any harmful or deleterious side effects.

A daily dosage of active ingredients can be expected to be about 1 to about 5000 milligrams per kilogram of body weight, with the preferred dose being about 10 to about 2000 mg/kg. Alternatively, the composition of the present invention is administered at a dose of about 50 to 1000 mg/kg. Alternatively, the composition of the present invention is administered at a dose of about 100 to 800 mg/kg. Alternatively, the composition of the present invention is administered at a dose of about 400 mg/kg once or twice daily.

Typically, the preparation of this invention will be administered from about 1 to about 5 times per day or alternatively, on an “as needed” basis for the topical formulation. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active composition (w/w). Preferably, such preparations contain from about 20% to about 80% active composition.

### Combination Therapy

Combination therapy is contemplated wherein the composition according to the invention is co-administered with at least one additional immuno-modulatory treatment. The additional agents may be combined with composition of this invention to create a single dosage form. Alternatively these additional agents may be separately administered, concurrently or sequentially, as part of a multiple dosage form.

When the pharmaceutical composition of this invention comprises a combination of a composition according to the invention, and one or more additional immuno-modulatory agent, both the composition and the additional agent should be present at dosage levels of between about 10 to 100%, and more preferably between about 10 and 80% of the dosage normally administered in a monotherapy regimen. In the case of a synergistic interaction between the composition of the invention and the additional immuno-modulatory agent or agents, the dosage of any or all of the active agents in the combination may be reduced compared to the dosage normally administered in a monotherapy regimen.

Immuno-modulatory agents contemplated for use in such combination therapy include agents (compounds or biologicals) that are effective to decrease the immune response in a mammal, including but not limited to agents that interfere with Th1 and/or Th2 responses in a mammal. Such agents can be selected from: corticosteroids, biologicals, immunomodulators and anti-inflammatory agents.

The present invention will be more readily understood by referring to the following examples which are given to illustrate the invention rather than to limit its scope.

### EXAMPLES

### Example 1

### Preparation of Composition

1000 kg of a whey protein isolate was re-hydrated up to 5% (50 g/L) in deionised water and stabilized overnight at 4° C. under agitation. The pH of the solution was adjusted to between 4.8 and 5.2 by addition of concentrated HCl. This adjustment induced protein precipitation. The precipitate was then recovered by centrifugation on a Wesfalia separator at 25 m³ per hour and washed with acidified water. The precipitate was re-hydrated to obtain 800 litres, which was thermally treated at 75° C. during 15 seconds, before concentration and spray-drying. Approximately 60 kg of composition was obtained.

### Example 2

### Analysis of the Composition

Typical analysis of the composition is presented in table 2. Proteins were analyzed by Kjeldahl Method and Dye Binding Method (AOAC Official Method 930.29; AOAC Official Method of Analysis (1995)), while fat content was quantified by the Roese-Gottieb Method (AOAC Official Method 932.06), humidity by drying in a dry oven, beta-lactoglobulin by RP-HPLC and growth factors by ELISA methods.

TABLE 2
Typical analysis of the composition

| Compound | Quantity (w/w) |
|---|---|
| Proteins (hydro-soluble proteins) | 90% (48%) |
| TGF-β1 | 0.3 μg/g of the composition |
| TGF-β2 | 11.7 μg/g of the composition |
| β-lactoglobulin | 66% of total proteins |
| IGF-1 | 40 μg/g of the composition |
| α-lactalbumin | 6% of total proteins |
| Fat | 1.9% |
| Humidity | 4.2% |
| Ashes | 2% |

### Example 3

### Inhibition of Proliferation of Mice Splenocytes and Cytokines Production

Female BALB/c mice, 6-8 weeks old, were obtained from Charles River (Montreal, Canada). Mice were sacrificed by CO₂ inhalation and single-cell suspensions were prepared individually from murine spleen under aseptic conditions. Murine lymphocytes (1.25×10⁶ cells/ml) were treated with ConA (1.25 μg/ml) for 72 hours. Alamar Blue® was added 24 hours before measuring ConA-induced lymphocytes proliferation. Cytokines (IL-4 & IL-10) levels and CD8 and CD8/CD25 proportions were also determined in the supernatant following the incubation period. BSA was used as a protein control (1-1000 μg/ml).

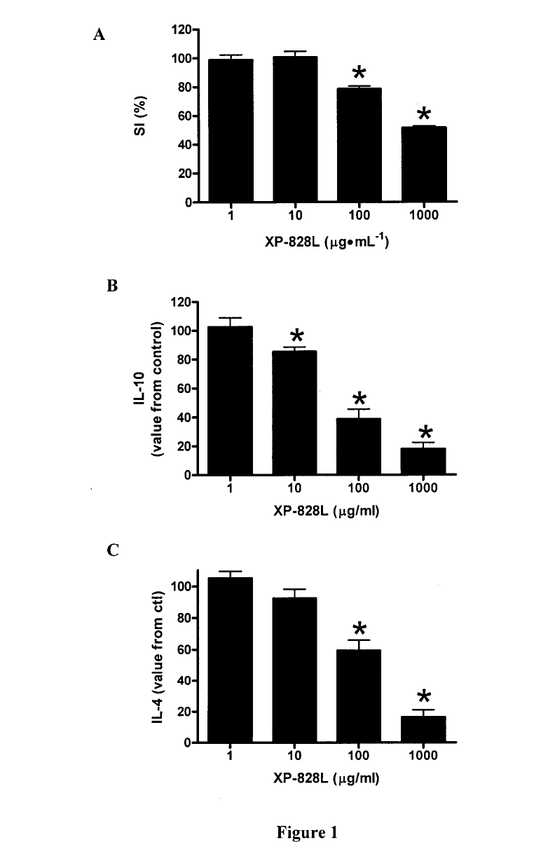

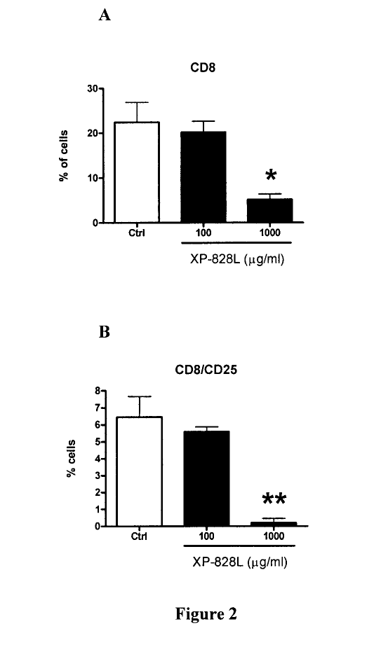

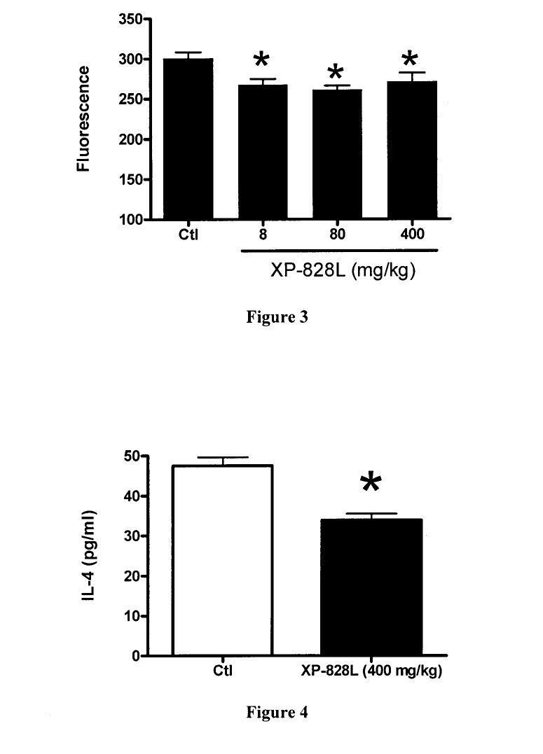

The composition (1, 10, 50, 100, 500, and 1000 μg of the total composition per ml of culture medium), was added to the cells for the whole incubation period and dose-dependently decreased 1) the lymphocyte proliferation (FIG. 1A) the production of IL-4 (FIG. 1C) the production of IL-10 (FIG. 1B). As shown, there was a significant inhibition (SI) of induced proliferation and Th2-related cytokines production by the composition. Furthermore, the CD8 proportion and CD8/CD25 ratio significantly decreased in presence of 1000 μg/ml of the composition according to example 2 (FIGS. 2A and 2B). Without wishing to be bound to theory, these results suggest that the composition presents immuno-modulatory properties and that it can be used to address immune-mediated chronic inflammatory diseases such as Type I (through the decreased of IL-4 production) and IV hypersensitivity disorders (through the decreased of T cell activity).

### Example 4

### In Vivo Immunomodulatory Properties of the Composition According to Example 2

Female BALB/c mice, 6-8 weeks old, were obtained from Charles River (Montreal, Canada) and treated by gavage with the composition according to example 2 at concentrations ranging from 8 to 400 mg/kg/day for 8 weeks. Following the treatment period of 8 weeks, mice were sacrificed by CO₂ inhalation and single-cell suspensions were prepared individually from murine spleen under aseptic conditions. Murine lymphocytes (1.25×10⁶ cells/ml) were treated with ConA (1.25 μg/ml) for 72 hours. Alamar Blue® was added 24 hours before measuring ConA-induced lymphocytes proliferation. Cytokines (IL-4) levels were also determined in the supernatant following the incubation period. BSA was used as a protein control (1-1000 μg/ml).

In vivo administration of the composition according to example 2 (400 mg/kg) for 8 weeks, significantly reduced ConA induced IL-4 cytokines by murine splenocytes (FIG. 4). These data suggest immunomodulatory properties of the composition according to example 2.

### Example 5

### Open Study on the Efficacy of the Composition According to Example 2 on Mild-to-Moderate Eczema Symptoms

Briefly, this study was open-labeled on the efficacy of the composition according to example 2 in mild-to-moderate atopic dermatitis (EASI score between 3 and 16) in adults weighting less than 100 kg.

Patients were asked to take 800 mg/day of the composition according to example 2 (oral form) for a period of 90 days. EASI score, DLQI, Patient's self-assessment score, Itching score and sleep quality was evaluated at day 1, 30, 60 and 90.

### Synopsis of Protocol

| | |
|---|---|
| PRODUCT | the composition according to example 2 |
| INDICATION | Atopic dermatitis mild to moderate |
| PATIENTS | Men or women of more than 18 years of age |
| | with stable atopic dermatitis (AD) mild to |
| | moderate (EASI [3-16]) and a body weight of |
| | less than 100 kg |
| | Subjects must have a diagnostic of AD on |
| | te basis of criterias from Hanifin et Rajka |
| | (15). |
| OBJECTIVES | Primary: |
| OF THE STUDY | Check efficacy of 800 mg/day of the |
| | composition according to example 2 in |
| | the treatment of atopic dermatitis mild |
| | to moderate (400 mg twice daily) |
| | Secondary: |
| | Accumulate data on quality of life of |
| | patients treated with the composition |
| | according to example 2. |
| DESIGN OF STUDY | Open labeled |
| POSOLOGICAL | Capsules |
| FORM | |
| ROUTE OF | Oral |
| ADMINISTRATION | |
| DURATION OF | 90 days |
| PARTICIPATION | |
| NUMBER OF | 15 |
| SUBJECTS | |
| NUMBER OF SITES | One |

### Results

### EASI Score

As shown in FIG. 5, at day 30, 4 out of 9 patients (44%) had a decreased EASI score (−0.2 to −2.3 units) compared to day 1. At day 60, 4 out of 8 patients (50%) had a decreased EASI score (−0.5 to −1.8). At day 90, 3 out of 5 patients (60%) had a decreased EASI score compared to day 1 (−0.7 to −2.4 units).

At any point in time (30, 60 or 90 days), 6 out of 9 patients (66%) had a decreased EASI score of at least 20% compared to day 0.

### DLQI Score

A decreased DLQI score reflects an increased quality of life.

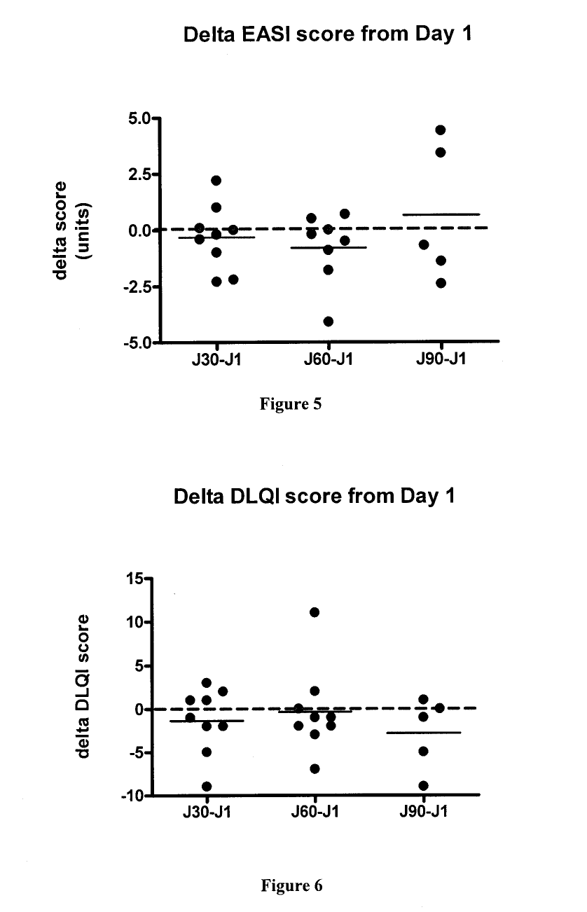

As shown in FIG. 6 at day 30, 5 out of 9 patients (56%) had a decreased DLQI score (−1 to −9 units) compared to day 1. At day 60, 6 out of 9 patients (67%) had a decreased DLQI score (−1 to −7). At day 90, 3 out of 5 patients (60%) had a decreased DLQI score compared to day 1 (−1 to −9 units).

Overall, these data are promising and strongly suggest that the composition according to example 2 is effective in improving the quality of life and diminishing the symptoms of mild to moderate eczema when administered orally in human subjects by way of two doses of 400 mg per day.

### REFERENCES

1. Janeway C A, Travers P, Walport M and Shlomchik M. Immunobiology—The immune system in health and disease. 5^{th} Edition, Garland Publishing, New-York 2001.
2. Cross M L and Gill H S. Modulation of immune function by modified bovine whey protein concentrate. Immunology and Cell Biology (1999) 77, 345-350.
3. Cross M L and Gill H S. Immunomodulatory properties of milk. British Journal of Nutrition (2000), 84 Suppl. 1, S81-89.
4. Marshall K. Therapeutic applications of whey protein. Alternative Medicine Review (2004), 9(2): 136-156.
5. Otani H, Monnai M. Inhibition of proliferative response of mouse spleen lymphocytes by bovine milk kappa-casein digests. Food Agric. Immunol. (1993) 5, 219-229.
6. Ontani H, Odashima M. Inhibition of proliferative responses of mouse spleen lymphocytes by lacto- and ovotransferrins. Food Agric Immunol. (1997) 9, 193-201.
7. Wong C W, Seow H F, Liu A H, Husband A J, Smithers G W, Watson D L. Modulation of immune responses by bovine beta-casein. Immunol. Cell Biol. (1996) 74, 323-329.
8. Wong C W, Seow H F, Husband A J, Regester G O, Watson D L. Effect of purified bovine whey factors on cellular immune functions in ruminants. Vet. Immunol. Immunopathol. (1997) 56, 85-96.
9. Kulkarni A B, Karlsson S. Transforming growth factor-beta 1 knockout mice. A mutation in one cytokine gene causes a dramatic inflammatory disease. Am J. Pathol. (1993) 143(1), 3-9.
10. Li M O, Wan Y Y, Sanjabi S, Robertson A-K L, and Flavell R A. Transforming growth factors-β regulation of immune responses. Annu. Rev. Immunol. (2006) 24, 99-146.
11. Kehrl J H, Wakefield L M, Roberts A B, Jakowlew S, Alvarez-Mon M, et al. Production of transforming growth factor B by human T lymphocytes and its potential role in the regulation of T cell growth. J. Exp. Med. (1986) 163, 1037-1350.
12. Gorelik L, Flavell R A. Transforming growth factor-beta in T-cell biology. Nat Rev Immunol. 2002, 2(1):46-53.
13. Maggie E., The Th1/Th2 paradigm in allergy. Immunotechnology (1998). 3(4):233-44.
14. Ro magnani S., T-cell subsets (Th1 versus Th2). Ann Allergy Asthma Immunol (2000). 85(1):9-18.
15. Hani fin J M, Rajka G. Acta Dermatol Venerol (Stockh) 1980; 92: 44-47.

1. A method for treating atopic eczema in a patient in need thereof, the method comprising administering to the patient an effective amount of a composition comprising 0.1 to 5 μg of TGF-β1 per gram of composition, between 5 to 50 μg of TGF-β2 per gram of composition, and at least 60% of β-lactoglobulin.

2. (canceled)

3. (canceled)

4. The method according to claim 1, wherein said TGF-β1 and TGF-β2 are derived from dairy sources, and are present at concentration between 0.2 to 1.2 μg/g and between 8 to 18 μg respectively per gram of composition.

5. (canceled)

6. The method according to claim 1, wherein said composition is administered orally or topically.

7. The method according to claim 6, wherein said composition is administered at a dosage of about 50 to 800 mg/kg.

8. The method according to claim 7, wherein said composition is administered orally at a dosage of about 800 mg per day.

9. (canceled)

10. (canceled)

11. (canceled)

12. (canceled)

13. (canceled)

14. (canceled)

15. (canceled)

16. A method for the treatment of atopic eczema in a human subject, comprising the steps of orally administering about 800 mg per day of a composition comprising:

| ingredient | Window |
|---|---|
| TGF-β2 | 8 μg/g to 20 μg/g; |
| TGF-β1 | 0.01 to 5 μg/g; |
| IGF-1 | 0.01 to 2 μg/g; |
| β-lactoglobulin (β-Lg) | 30% to 85%; |
| α-lactalbumin (α-Lac) | 0% to 25%; |
| Lactoferrin (Lf) | 0% to 15%; and |
| Immunoglobulin (IgG) | 2% to 15%. |

17. The method according to claim 16, wherein the composition essentially consists of:

| | |
|---|---|
| Whey proteins | 90% |
| TGF-β1 | 0.3 μg/g of the composition; |
| TGF-β2 | 11.7 μg/g of the composition; |
| β-lactoglobulin | 66% of total proteins; |
| IGF-1 | 40 μg/g of the composition; and |
| α-lactalbumin | 6% of total proteins. |

18. (canceled)

19. (canceled)

20. (canceled)

21. (canceled)
